(19) United States
(12) Patent Application Publication
(10) Pub. No.: US 2025/0099751 A1
(43) Pub. Date: Mar. 27, 2025

(54) PANCREATIC CANCER TREATMENT SYSTEMS AND METHODS

(71) Applicant: Boston Scientific Scimed, Inc., Maple Grove, MN (US)

(72) Inventors: Brian L. Schmidt, White Bear Lake, MN (US); Devon N. Arnholt, Shoreview, MN (US); Michael J. Lyden, Shoreview, MN (US); Michael J. Kane, St. Paul, MN (US); Tucker James Nelson, Bloomington, MN (US)

(73) Assignee: MAYO FOUNDATION FOR MEDICAL EDUCATION AND RESEARCH, Rochester, MN (US)

(21) Appl. No.: 18/893,371

(22) Filed: Sep. 23, 2024

Related U.S. Application Data
(60) Provisional application No. 63/540,146, filed on Sep. 25, 2023.

(51) Int. Cl. A61N 1/36; A61N 1/05; A61N 1/08; A61N 1/375

(52) U.S. Cl. CPC A61N 1/36002; A61N 1/056; A61N 1/3756; A61N 1/08

(57) ABSTRACT

Embodiments herein relate to implantable systems and methods for treatment of pancreatic cancer. In an embodiment, a method of treating pancreatic cancer is included, the method including inserting an electrical stimulation lead through the inferior vena cava, a hepatic vein, and into the portal vein via a transjugular intrahepatic portosystem shunt (TIPS). The method can further include inserting the electrical stimulation lead into at least one of the superior mesenteric vein and the splenic vein. The method can further include positioning electrodes on the lead within at least one of the superior mesenteric vein and the splenic vein and delivering one or more electric fields through the electrodes to a treatment zone including at least a portion of the pancreas. The electric fields can be at frequencies and a field strength effective to prevent and/or disrupt cellular mitosis in a cell.

This application claims the benefit of U.S. Provisional Application No. 63/540,146, filed Sep. 25, 2023, the content of which is herein incorporated by reference in its entirety.

### FIELD

Embodiments herein relate to implantable systems for cancer treatment and related methods. More specifically, embodiments herein relate to implantable systems and methods for treatment of pancreatic cancer.

### BACKGROUND

According to the American Cancer Society, cancer accounts for nearly 25% of the deaths that occur in the United States each year. Cancerous tumors can form if one normal cell in any part of the body mutates and then begins to grow and multiply too much and too quickly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division.

Surgery is a common first-line therapy for many cancerous tumors. However, not every tumor can be surgically removed. Pancreatic cancer is a type of cancer that originates in the pancreas, an organ that produces digestive enzymes and hormones such as insulin. Pancreatic cancer can be difficult to detect and treat because it often does not cause symptoms until it has spread limiting treatment options. For example, ingrowth into the superior mesenteric artery or celiac axis can cause the tumor to be unresectable, substantially limiting treatment options.

### SUMMARY

Embodiments herein relate to implantable systems and methods for treatment of pancreatic cancer. In a first aspect, a method of treating pancreatic cancer is included, the method including inserting an electrical stimulation lead through the inferior vena cava, a hepatic vein, and into the portal vein via a transjugular intrahepatic portosystem shunt (TIPS). The method can further include inserting the electrical stimulation lead into at least one of the superior mesenteric vein and the splenic vein. The method can further include positioning electrodes connected to the lead within at least one of the superior mesenteric vein and the splenic vein and delivering one or more electric fields through the electrodes to a treatment zone including at least a portion of the pancreas. The electric fields can be at frequencies and a field strength effective to prevent and/or disrupt cellular mitosis in a cell.

In a second aspect, in addition to one or more of the preceding or following aspects, or in the alternative to some aspects, the method can further include implanting a medical device configured to generate the one or more electrical fields. The medical device can further include control circuitry and a therapy output circuit. The method can further include attaching the electrical stimulation lead to the medical device.

In a third aspect, in addition to one or more of the preceding or following aspects, or in the alternative to some aspects, the medical device can further include a conductive housing, wherein the conductive housing can be configured to serve as an electrode for use with delivering the one or more electrical fields.

In a fourth aspect, in addition to one or more of the preceding or following aspects, or in the alternative to some aspects, inserting the electrical stimulation lead can include inserting a first electrical stimulation lead into the superior mesenteric vein and inserting a second electrical stimulation lead into the splenic vein.

In a fifth aspect, in addition to one or more of the preceding or following aspects, or in the alternative to some aspects, the one or more electric fields can be at a field strength of 1 V/cm to 10 V/cm within a treatment zone.

In a sixth aspect, in addition to one or more of the preceding or following aspects, or in the alternative to some aspects, the method can further include selecting a patient with an unresectable pancreatic tumor.

In a seventh aspect, in addition to one or more of the preceding or following aspects, or in the alternative to some aspects, the method can further include selecting a patient with tumor ingrowth into the superior mesenteric artery or celiac axis.

In an eighth aspect, in addition to one or more of the preceding or following aspects, or in the alternative to some aspects, at least one the electrodes can have an axial length from 1.5 to 2.5 centimeters.

In a ninth aspect, in addition to one or more of the preceding or following aspects, or in the alternative to some aspects, at least one the electrodes can have a diameter of 0.8 to 1.2 millimeters.

In a tenth aspect, in addition to one or more of the preceding or following aspects, or in the alternative to some aspects, the method can further include selecting one or more pairs of electrodes forming a vector across which to deliver electrical fields.

In an eleventh aspect, a method of treating pancreatic cancer can be included. The method can include inserting an electrical stimulation lead into the inferior vena cava. The method can further include positioning electrodes on the lead within the inferior vena cava and adjacent to the pancreas. The method can further include delivering one or more electric fields through the electrodes to a treatment zone including at least a portion of the pancreas. The one or more electric fields can be at frequencies and field strengths effective to prevent and/or disrupt cellular mitosis in a cell.

In a twelfth aspect, in addition to one or more of the preceding or following aspects, or in the alternative to some aspects, can further include implanting a medical device configured to generate the one or more electrical fields, the medical device can include control circuitry and a therapy output circuit. The method can further include attaching the electrical stimulation lead to the medical device.

In a thirteenth aspect, in addition to one or more of the preceding or following aspects, or in the alternative to some aspects, the medical device can further include a conductive housing, wherein the conductive housing can be configured to serve as an electrode for use with delivering the one or more electrical fields.

In a fourteenth aspect, in addition to one or more of the preceding or following aspects, or in the alternative to some aspects, the one or more electric fields can be at a field strength of 1 V/cm to 10 V/cm within the treatment zone.

In a fifteenth aspect, in addition to one or more of the preceding or following aspects, or in the alternative to some aspects, can further include inserting a second electrical stimulation lead transversely across the abdomen, the second electrical stimulation lead can include a second set of electrodes disposed thereon.

In a sixteenth aspect, in addition to one or more of the preceding or following aspects, or in the alternative to some aspects, the method can further include selecting a patient with an unresectable pancreatic tumor.

In a seventeenth aspect, in addition to one or more of the preceding or following aspects, or in the alternative to some aspects, the method can further include selecting a patient with tumor ingrowth into the superior mesenteric artery or celiac axis.

In an eighteenth aspect, in addition to one or more of the preceding or following aspects, or in the alternative to some aspects, wherein at least one the electrodes can have an axial length from 1.5 to 2.5 centimeters.

In a nineteenth aspect, in addition to one or more of the preceding or following aspects, or in the alternative to some aspects, wherein at least one the electrodes can have a diameter of 0.8 to 1.2 millimeters.

In a twentieth aspect, in addition to one or more of the preceding or following aspects, or in the alternative to some aspects, the method can further include selecting one or more pairs of electrodes forming a vector across which to deliver electrical fields.

In a twenty-first aspect, a method of treating pancreatic cancer can be included. The method can include inserting electrical stimulation electrodes into a patient, positioning the electrodes into a ductal system of a pancreas of the patient, and delivering one or more electric fields through the electrodes to a treatment zone including at least a portion of the pancreas. The one or more electric fields can be at frequencies selected from a range of between 10 kHz to 1 MHz, wherein the electric fields can be effective to prevent and/or disrupt cellular mitosis in a cell.

In a twenty-second aspect, in addition to one or more of the preceding or following aspects, or in the alternative to some aspects, the method can further include positioning the electrodes in the main pancreatic duct of the pancreas of the patient.

In a twenty-third aspect, in addition to one or more of the preceding or following aspects, or in the alternative to some aspects, the method can further include inserting the electrodes through at least one of the minor duodenal papilla and the major duodenal papilla of the patient.

In a twenty-fourth aspect, in addition to one or more of the preceding or following aspects, or in the alternative to some aspects, the method can further include implanting a medical device configured to generate the one or more electrical fields and attaching the electrodes to the medical device. The medical device can include control circuitry and a therapy output circuit.

In a twenty-fifth aspect, in addition to one or more of the preceding or following aspects, or in the alternative to some aspects, the medical device further can include a conductive housing, wherein the conductive housing can be configured to serve as an electrode for use with delivering the one or more electrical fields.

In a twenty-sixth aspect, in addition to one or more of the preceding or following aspects, or in the alternative to some aspects, the one or more electric fields can be at a field strength of 1 V/cm to 10 V/cm within the treatment zone.

In a twenty-seventh aspect, in addition to one or more of the preceding or following aspects, or in the alternative to some aspects, at least one of the electrodes can have an axial length from 1.5 to 2.5 centimeters.

In a twenty-eighth aspect, in addition to one or more of the preceding or following aspects, or in the alternative to some aspects, at least one of the electrodes can have a diameter of 0.8 to 1.2 millimeters.

In a twenty-ninth aspect, in addition to one or more of the preceding or following aspects, or in the alternative to some aspects, the method can further include selecting one or more pairs of electrodes forming a vector across which to deliver electrical fields.

In a thirtieth aspect, a method of treating pancreatic cancer can be included. The method can include inserting electrical stimulation electrodes into a patient, directly positioning the electrodes into or adjacent to a pancreas of the patient, and delivering one or more electric fields through the electrodes to a treatment zone including at least a portion of the pancreas. The one or more electric fields can be at frequencies selected from a range of between 10 kHz to 1 MHz, wherein the electric fields can be effective to prevent and/or disrupt cellular mitosis in a cell.

In a thirty-first aspect, in addition to one or more of the preceding or following aspects, or in the alternative to some aspects, the method can further include positioning the electrodes outside of the veinous system and outside of the ductal system of the pancreas of the patient.

In a thirty-second aspect, in addition to one or more of the preceding or following aspects, or in the alternative to some aspects, the method can further include implanting a medical device configured to generate the one or more electrical fields and attaching the electrodes to the medical device. The medical device can include control circuitry and a therapy output circuit.

In a thirty-third aspect, in addition to one or more of the preceding or following aspects, or in the alternative to some aspects, the medical device can further include a conductive housing, wherein the conductive housing can be configured to serve as an electrode for use with delivering the one or more electrical fields.

In a thirty-fourth aspect, in addition to one or more of the preceding or following aspects, or in the alternative to some aspects, the one or more electric fields can be at a field strength of 1 V/cm to 10 V/cm within the treatment zone.

In a thirty-fifth aspect, in addition to one or more of the preceding or following aspects, or in the alternative to some aspects, at least one of the electrodes can have an axial length from 1.5 to 2.5 centimeters.

In a thirty-sixth aspect, in addition to one or more of the preceding or following aspects, or in the alternative to some aspects, at least one of the electrodes can have a diameter of 0.8 to 1.2 millimeters.

In a thirty-seventh aspect, in addition to one or more of the preceding or following aspects, or in the alternative to some aspects, the method can further include selecting one or more pairs of electrodes forming a vector across which to deliver electrical fields.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a simplified schematic view of the pancreas in accordance with various embodiments herein.

FIG. 2 is a simplified schematic view of a portion of anatomy including the pancreas in accordance with various embodiments herein.

FIG. 3 is a schematic view of a lead insertion pathway in accordance with various embodiments herein.

FIG. 4 is a schematic view of electrical stimulation lead and electrode in accordance with various embodiments herein.

FIG. 5 is a schematic view of electrical stimulation lead placement in accordance with various embodiments herein.

FIG. 6 is a schematic view of electrical stimulation lead placement in accordance with various embodiments herein.

FIG. 7 is a schematic view of electrical stimulation lead placement in accordance with various embodiments herein.

FIG. 8 is a schematic view of a cancer therapy lead in accordance with various embodiments herein.

FIG. 9 is a schematic cross-sectional view of a cancer therapy lead in accordance with various embodiments herein.

FIG. 10 is a schematic cross-sectional view of a medical device in accordance with various embodiments herein.

FIG. 11 is a schematic diagram of components of a medical device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Pancreatic cancer can be difficult to detect and treat because it often does not cause symptoms until it has spread, thereby limiting treatment options. For example, ingrowth into the superior mesenteric artery or celiac axis can cause the tumor to be deemed unresectable, substantially limiting treatment options.

However, embodiments of system and methods herein can be used to treat pancreatic cancer including, but not limited to, unresectable pancreatic cancer tumors. In an embodiment, a method of treating pancreatic cancer herein can include inserting an electrical stimulation lead through the inferior vena cava and then a hepatic vein, and into the portal vein via a transjugular intrahepatic portosystem shunt (TIPS). Then the electrical stimulation lead can be inserted into at least one of the superior mesenteric vein and the splenic vein. The method can further include positioning electrodes on the lead into at least one of the superior mesenteric vein and the splenic vein and delivering one or more electric fields through the electrodes to a treatment zone including at least a portion of the pancreas. The electric fields can be at frequencies and field strengths that are effective to prevent and/or disrupt cellular mitosis in a cell, but less than that used for tissue ablation. Further details regarding the electric fields are provided below.

In various other embodiments, a method of treating pancreatic cancer herein can include inserting an electrical stimulation lead into the inferior vena cava, positioning electrodes on the lead within the inferior vena cava and adjacent to the pancreas, and delivering one or more electric fields through the electrodes to a treatment zone including at least a portion of the pancreas. In some embodiments, another electrical stimulation lead can be inserted transversely across the abdomen and the electrodes thereon can be used in combination with electrodes of the first electrical stimulation lead and/or conductive housings of implanted devices serving as electrodes to deliver therapy.

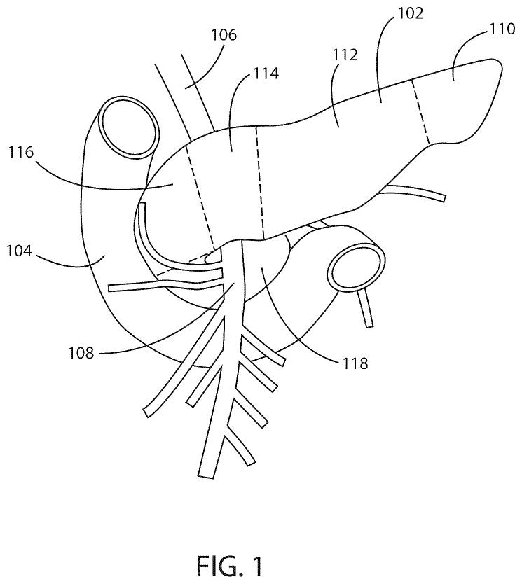

Referring now to FIG. 1, a simplified schematic view of the pancreas 102 is shown in accordance with various embodiments herein. Along with the pancreas 102, the superior mesenteric vein 108 is shown. In this view, the duodenum 104 of a patient is also shown along with a portal vein 106. The pancreas 102 can be divided into various portions. For example, the pancreas 102 includes the tail 110, body 112, neck 114, head 116, and uncinate process 118.

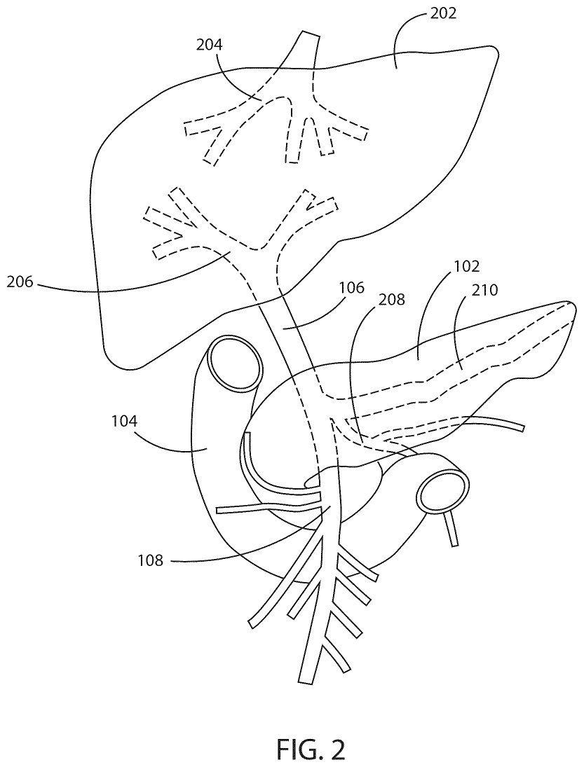

Referring now to FIG. 2, a simplified schematic view of a portion of anatomy including the pancreas 102 is shown in accordance with various embodiments herein. As before, along with the pancreas 102, the superior mesenteric vein 108, duodenum 104, and portal vein 106 are shown. Additional anatomical features shown in this view include an inferior mesenteric vein 208, splenic vein 210, and liver 202. The liver 202 is shown along with portions of the hepatic veinous system 204 and portions of the hepatic portal system 206. Various of these anatomical features can be utilized in order to insert an electrical stimulation lead into a position so as to allow for treatment of a targeted zone that can be within or include at least a portion of the pancreas 102.

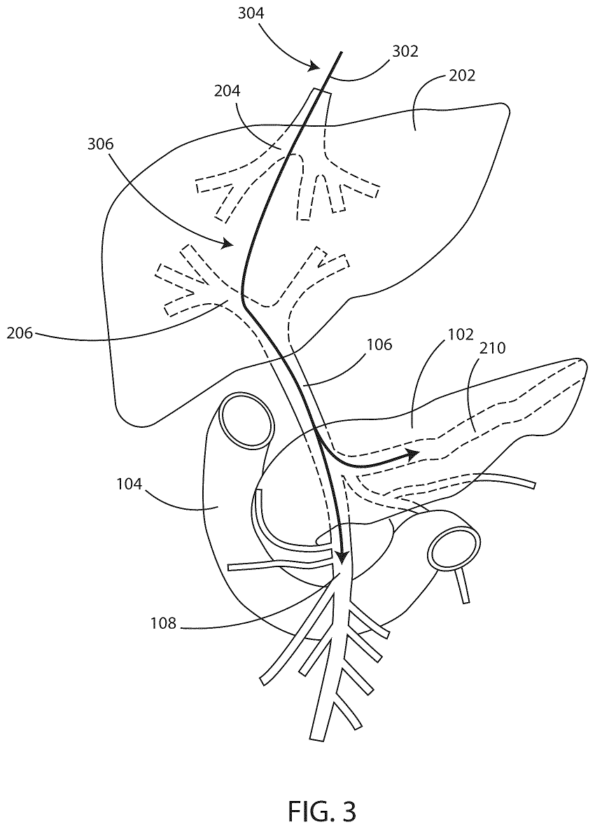

Referring now to FIG. 3, a schematic view of an exemplary lead insertion pathway 302 is shown in accordance with various embodiments herein. As before, the pancreas 102 is shown along with the superior mesenteric vein 108, duodenum 104, portal vein 106, inferior mesenteric vein 208, splenic vein 210, and liver 202. The liver 202 is shown along with portions of the hepatic veinous system 204 and portions of the hepatic portal system 206. The lead insertion pathway 302 can include the inferior vena cava 304, the hepatic veinous system 204, a transjugular intrahepatic portosystem shunt (TIPS) 306, the hepatic portal system 206, portal vein 106, and then into splenic vein 210 and/or superior mesenteric vein 108. The inferior vena cava 304 can be surgically accessed in various ways including, but not limited to, jugular access and femoral access. In various embodiments, a stent-graft can be placed as part of forming the transjugular intrahepatic portosystem shunt 306.

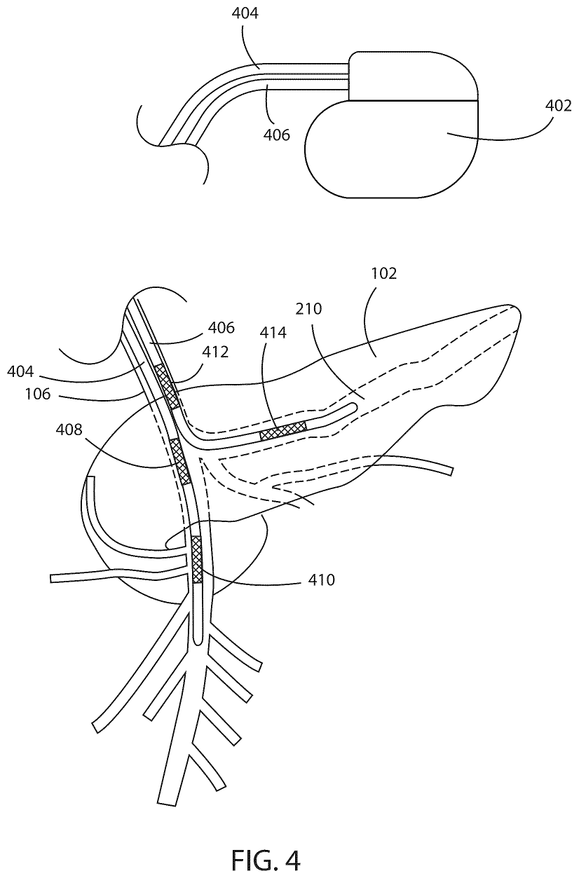

In various embodiments, a first electrical stimulation lead can be inserted into the superior mesenteric vein 108 and a second electrical stimulation lead can be inserted into the splenic vein 210. Referring now to FIG. 4, a schematic view of electrical stimulation leads and electrodes is shown in accordance with various embodiments herein. The pancreas 102 is shown along with a splenic vein 210 and a portal vein 106. FIG. 4 shows an implantable medical device 402. Attached to the implantable medical device 402, FIG. 4 also shows a first electrical stimulation lead 404 including a first electrode 408 and a second electrode 410 (though it will be appreciated that various numbers of electrodes can be used on an electrical stimulation lead such as 1, 2, 3, 4, 4, 6, etc.). FIG. 4 shows a second electrical stimulation lead 406 which includes a third electrode 412 and a fourth electrode 414 disposed thereon. Pairs of electrodes can be used to form vectors across which electrical fields are delivered to a treatment site including at least a portion of the pancreas 102. In some embodiments, at least one electrode can be positioned at least slightly above a mid-point of a target therapy zone (such as a position of a tumor to be treated). In some embodiments, at least one electrode can be positioned at least slightly below a mid-point of a target therapy zone (such as a position of a tumor to be treated). In some embodiments, at least one electrode can be positioned so that it is substantially centered within the target therapy zone (such as a position of a tumor to be treated).

The implantable medical device 402 further can include a conductive housing, wherein the conductive housing can be configured to serve as an electrode for use with delivering the one or more electrical fields. Further details of exemplary implantable medical devices are provided in greater detail below.

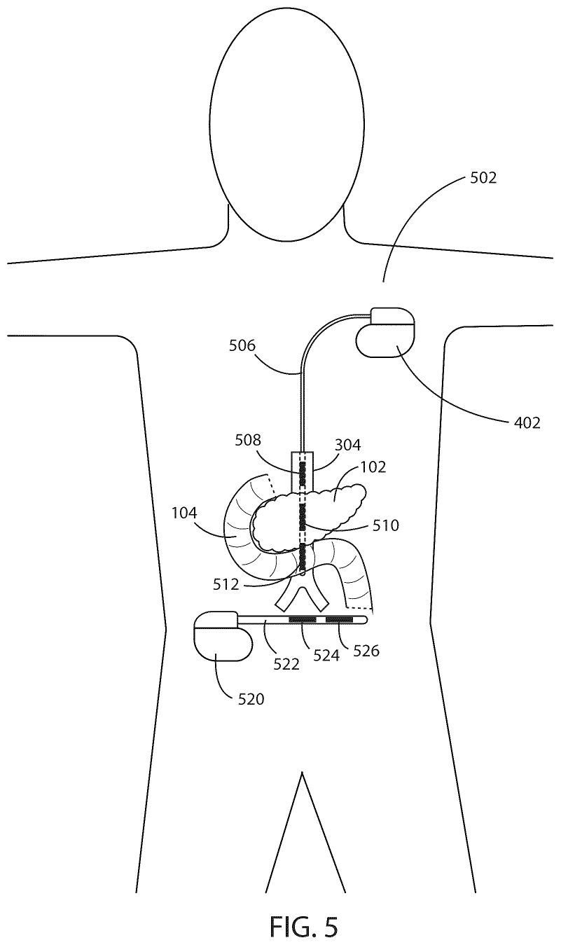

Other positioning for electrical stimulation leads in order to treat pancreatic cancer are also contemplated herein. For example, referring now to FIG. 5, a schematic view of electrical stimulation lead placement is shown in accordance with various embodiments herein. FIG. 5 shows a patient 502 along with an implantable medical device 402. The pancreas 102 of the patient 502 is schematically shown along with the duodenum 104 and the inferior vena cava 304. In this example, the electrical stimulation lead 506 includes a first electrode 508, a second electrode 510, and a third electrode 512. Pairs of electrodes can be used to form vectors across which electrical fields are delivered to a treatment site including at least a portion of the pancreas 102. The positioning with in the inferior vena cava 304 shown in FIG. 5 provides a substantially vertical orientation to the electrodes. In some embodiments, at least one electrode (such as first electrode 508) can be positioned at least slightly above a mid-point of a target therapy zone (such as a position of a tumor to be treated). In some embodiments, at least one electrode (such as third electrode 512) can be positioned at least slightly below a mid-point of a target therapy zone (such as a position of a tumor to be treated). In some embodiments, at least one electrode (such as second electrode 510) can be positioned so that it is substantially centered vertically within the target therapy zone.

In some embodiments, a second electrical stimulation lead can be implanted transversely across the abdomen, such as subcutaneously outside of the anterior abdominal muscles, inside of the abdominal cavity, etc. As such, FIG. 5 shows a second implantable device 520 along with a transverse abdominal electrical stimulation lead 522. The transverse abdominal electrical stimulation lead 522 also includes a first abdominal electrode 524 and a second abdominal electrode 526. Pairs of electrodes (on one or both leads) can be used to form vectors across which electrical fields are delivered to a treatment site including at least a portion of the pancreas 102. In some embodiments, at least one electrode is disposed to one lateral side of a target therapy zone (such as a position of a tumor to be treated) and at least one electrode is disposed to the other lateral side of the target therapy zone.

In some embodiments, a single implantable device can be used, such that in this example both electrical stimulation leads can be connected to the same implantable medical device (e.g., one of medical device 402 and second implantable device 520 can be omitted with both leads connected to the remaining device). As such, placement of medical device 402 and second implantable device 520 can be thought of as alternatives in some embodiments. In addition, in some embodiments, only a single electrical stimulation lead (506 or 522) can be used as connected to one of the implantable devices (402 or 520).

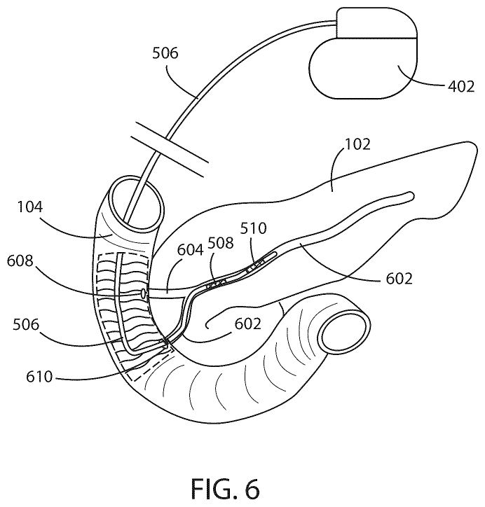

In some embodiments, the ductal system of the pancreas can be used for the positioning of electrical stimulation leads herein. Referring now to FIG. 6, a schematic view of electrical stimulation lead placement in accordance with various embodiments herein. In the view shown in FIG. 6, the pancreas 102 is shown along with duodenum 104. The pancreas 102 is shown along with main pancreatic duct 602 and accessory pancreatic duct 604. The duodenum 104 is shown along with minor duodenal papilla 608 and major duodenal papilla 610.

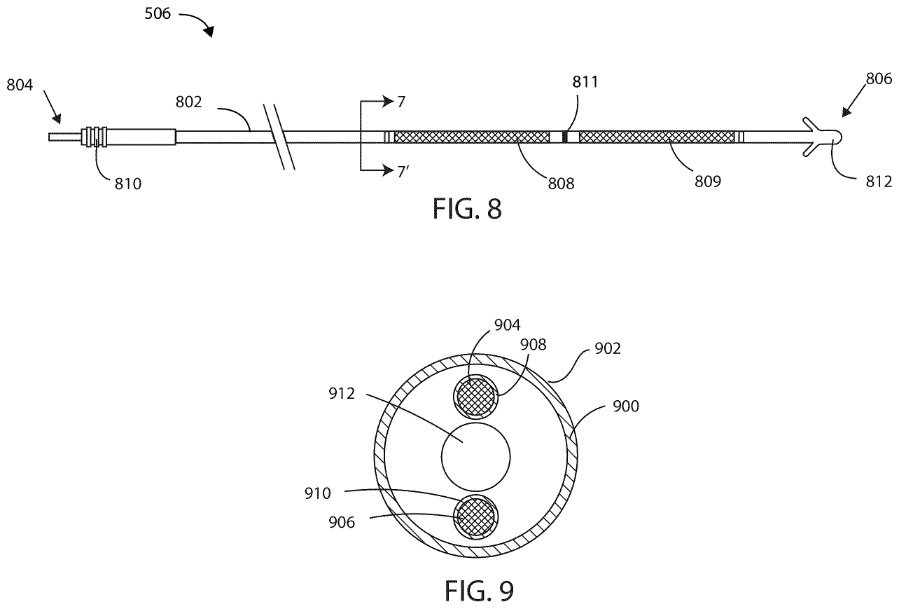

In this embodiment, there is a lead 506 that is connected to medical device 402. The lead 506 passes through the interior of the duodenum 104 and into major duodenal papilla 610. Access to the major duodenal papilla 610 can be achieved using techniques similar to that used with ERCP (endoscopic retrograde cholangiopancreatography). The lead 506 then passes through main pancreatic duct 602. First electrode 508 and second electrode 510 on lead 506 are positioned within main pancreatic duct 602 in order to treat a site (such as a tumor) within or adjacent to pancreas 102. However, it will be appreciated that the leads and/or electrodes thereof can be disposed within any portion of the ductal system of the pancreas. Further, in some embodiments, the minor duodenal papilla 608 can be used instead or in addition to the major duodenal papilla 610 (in some embodiments, one lead can be inserted into minor duodenal papilla 608 and a second lead can be inserted into major duodenal papilla 610). In some embodiments, the electrode and/or a portion of the lead can take the form of a stent or other expanding structure to allow the duct to remain open while also allowing the electrode to remain in position for treatment. Electrodes of other embodiments herein can also take the form of a stent or stent like structure. Also, it will be appreciated that leads herein (such as lead 506 in the context of FIG. 6, but also applicable to other embodiments herein) can include structure that is sufficient to electrically connect therapy electrode with a device to generate the electrical therapy, such as medical device 402. For example, the lead 506 can be as simple as one or more conductors (such as one or more insulated wires) in some embodiments, but in other embodiments can include a more complex structure such as that depicted with regard to FIG. 8 herein.

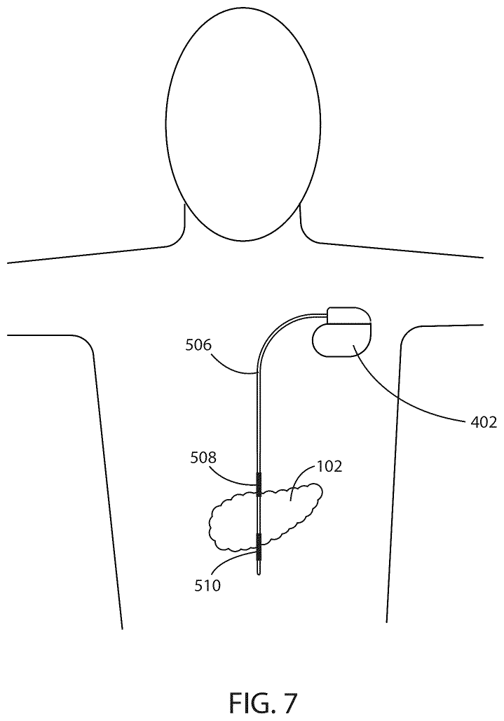

In some embodiments, electrical stimulation leads herein can be directed placed/inserted into a desired site and avoiding critical structures and/or ducts and veins. For example, a plunge lead/electrode(s) can be used to place the electrodes of a lead at a desired site to treat a site (such as a tumor) within or adjacent to pancreas 102. Referring now to FIG. 7, a schematic view of electrical stimulation lead placement in accordance with various embodiments herein. In this embodiment, lead 506 is connected to medical device 402. The lead 506 passes to a desired treatment site such that first electrode 508 and second electrode 510 on lead 506 are positioned properly to treat a site (such as a tumor) within or adjacent to pancreas 102. While two electrodes are shown in the example of FIG. 7, it will be appreciated that different numbers of electrodes can also be used. The electrodes can take the form of plunge electrodes and can be inserted such as using a catheter and/or insertion needle, such as with a lead over approach. The leads can be tunneled in from various points, such as being tunneled in from the abdomen. In various embodiments, multiple leads can be used. By way of example, multiple leads can be used to surround and/or triangulate around a desired treatment site, such as a tumor.

### Cancer Therapy Stimulation Leads

Referring now to FIG. 8, a schematic view of an exemplary cancer therapy stimulation lead 506 is shown in accordance with various embodiments herein. The cancer therapy stimulation lead 506 can include a lead body 802 with a proximal end 804 and a distal end 806. In this example, a first electrode 808 and a second electrode 809 are coupled to the lead body 802, as positioned near a distal end 806 thereof. In some embodiments, the electrodes 808, 809 can include electric field generating electrodes (which can function as working electrodes or counter electrodes depending on the system configuration). In various embodiments, the electrodes 808, 809 can include electric field sensing electrodes. The electrodes 808, 809 can be internally connected or internally independent. The lead body 802 can define a lumen. The electrodes 808, 809 can include various conductive materials such as platinum, silver, gold, iridium, titanium, and various alloys. In some embodiments, the cancer therapy stimulation lead 506 includes more than two electrodes. The electrodes can be of various sizes. In some embodiments, at least one of the electrodes has a diameter of 0.8 to 1.2 millimeters. In some embodiments, at least one the electrodes has an axial length from 1.5 to 2.5 centimeters.

In some embodiments, the cancer therapy stimulation lead 506 can include one or more therapy zone temperature sensors disposed along a length of the cancer therapy stimulation lead. In this example, a therapy zone temperature sensor 811 is positioned between the first electrode 808 and the second electrode 809. However, the therapy zone temperature sensor 811 can also be positioned at various other points along or in the lead. In some embodiments, the therapy zone temperature sensor 811 can be positioned in the lead directly beneath an electrode. The therapy zone temperature sensor 811 can include an optical or electrical thermal sensor. For example, the therapy zone temperature sensor can include a thermistor. The therapy zone temperature sensor 811 can be used to measure the thermal heating about the cancer therapy stimulation lead to provide feedback to a clinician about the local thermal heating zone around the lead and provide a tissue temperature of the treatment site to the medical device.

In various embodiments, the therapy zone temperature sensor 811 can provide a tissue temperature at a site offset from a surface of the electrodes 808, 809. If a tissue temperature of a site offset from the electrodes 808, 809 is measured, the medical device can compensate for the offset when measuring or estimating the temperature of the tissue. In some embodiments, the therapy zone temperature sensor 811 can measure or estimate the reference or core body temperature of the patient when the therapy is turned off or paused. While not intending to be bound by theory, in some scenarios it can be easier to get an accurate measurement of a reference or core body temperature when therapy is turned off or paused. In some embodiments, therapy zone temperature sensor data can be recorded and relayed to the clinician, patient, care provider, and/or medical record system.

The cancer therapy stimulation lead 506 can further include a terminal pin 810 for connecting the cancer therapy stimulation lead 506 to a medical device, such as a cancer treatment device. The terminal pin 810 can be compatible with various standards for lead-header interface design including the DF-1, VS-1, IS-1, LV-1 and IS-4 standards, amongst other standards.

In some embodiments, the cancer therapy stimulation lead 506 can further include a fixation element 812, such as an element that can adhere to a portion of the subject's body to maintain the position of the cancer therapy stimulation lead 506 and/or the electrodes 808. In various embodiments, the fixation element 812 can be disposed along the distal end 806 of the cancer therapy stimulation lead 506. However, in some embodiments a fixation element 812 is omitted.

Referring now to FIG. 9, a cross-sectional schematic view of a cancer therapy stimulation lead 506 as taken along line 7-7′ of FIG. 8 is shown in accordance with various embodiments herein. The cancer therapy stimulation lead 506 can include an outer layer 900 with an outer surface 902. The outer layer 900 can be flexible and can be configured to protect other components disposed within the lumen of the outer layer 900. In some embodiments, the outer layer 900 can be circular in cross-section. In some embodiments, the outer layer 900 includes a dielectric material and/or an insulator. In some embodiments, the outer layer 900 can include various biocompatible materials such as polysiloxanes, polyethylenes, polyamides, polyurethane and the like.

In various embodiments, the cancer therapy stimulation lead 506 can include one or more conductors, such as a first conductor 904 and a second conductor 906. It will be appreciated that this is only by way of example and that the actual number of conductors within a lead can far exceed two. In some embodiments, the first conductor 904 and the second conductor 906 can be disposed within the lumen of the outer layer 900. The first conductor 904 and a second conductor 906 can be configured to provide electrical communication between an electrode 808 and the proximal end 804 of the cancer therapy stimulation lead 506. The first conductor 904 and a second conductor 906 can include various materials including copper, aluminum, silver, gold, and various alloys such as tantalum/platinum, MP35N and the like. An insulator 908 and 910 can surround the first conductor 904 and a second conductor 906. The insulators 908 and 910 can include various materials such as electrically insulating polymers.

In some embodiments, each of the electrodes 808 can have individual first conductors 904 and second conductors 906 to electrically couple the electrode 808 to the proximal end 804 of the cancer therapy stimulation lead 506. However, in some embodiments, each of the electrodes 808 only connects to a single conductor to electrically couple the electrode 808 to the proximal end 804 of the cancer therapy stimulation lead 506. In some embodiments, the first conductor 904 and a second conductor 906 can be configured as a coil or a cable. Multiple conductors can be disposed within the lumen of the outer layer 900. For example, a separate conductor or set of conductors can be in communication with each electrode disposed along the lead. In various embodiments, a first conductor 904 and a second conductor 906 can form a part of an electrical circuit by which the electric fields from the electric field generating circuit are delivered to the site of the cancerous tissue. Many more conductors than are shown in FIG. 9 can be included within embodiments herein. For example, the cancer therapy stimulation lead 506 can include 1, 2, 3, 4, 5, 6, 7, 8, 10, 15 or 20 or more conductors, or any number of conductors falling within a range between any of the foregoing.

In some embodiments, the cancer therapy stimulation lead 506 can include a central channel 912. The central channel 912 can be configured for a guide wire, or other implanting device, to pass through, such as to aid in implanting the cancer therapy stimulation lead 506 and electrodes 808. In some cases, additional channels (not shown) are disposed within the cancer therapy stimulation lead 506.

### Medical Device Components

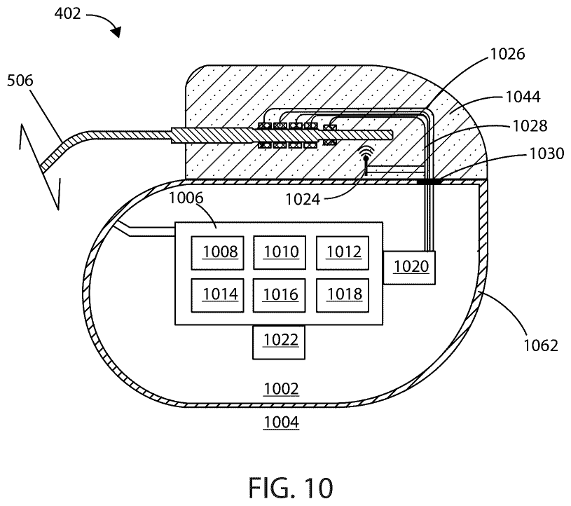

Referring now to FIG. 10, a schematic cross-sectional view of an exemplary medical device 402 is shown in accordance with various embodiments herein. The housing 1062 can define an interior volume 1002 that can be hollow and that in some embodiments is hermetically sealed off from the area 1004 outside of medical device 402. In other embodiments the housing 1062 can be filled with components and/or structural materials such that it is non-hollow. The medical device 402 can include control circuitry 1006, which can include various components 1008, 1010, 1012, 1014, 1016, and 1018 disposed within housing 1062. In some embodiments, these components can be integrated and in other embodiments these components can be separate. In yet other embodiments, there can be a combination of both integrated and separate components. The medical device 402 can also include an antenna 1024, to allow for unidirectional or bidirectional wireless data communication, such as with an external device or an external power supply. In some embodiments, the components of medical device 402 can include an inductive energy receiver coil (not shown) communicatively coupled or attached thereto to facilitate transcutaneous recharging of the medical device via recharging circuitry.

The various components 1008, 1010, 1012, 1014, 1016, and 1018 of control circuitry 1006 can include, but are not limited to, a microprocessor, memory circuit (such as random access memory (RAM) and/or read only memory (ROM)), recorder circuitry, controller circuit, a telemetry circuit, a power supply circuit (such as a battery), a timing circuit, and an application specific integrated circuit (ASIC), a recharging circuit, amongst others. Control circuitry 1006 can be in communication with an electric field generating circuit 1020 that can be configured to generate electric current to create one or more fields. The electric field generating circuit 1020 can be integrated with the control circuitry 1006 or can be a separate component from control circuitry 1006. Control circuitry 1006 can be configured to control delivery of electric current from the electric field generating circuit 1020. In some embodiments, the electric field generating circuit 1020 can be present in a portion of the medical device that is external to the body.

In some embodiments, the control circuitry 1006 can be configured to direct the electric field generating circuit 1020 to deliver an electric field via leads 506 and electrodes theron to the site of a cancerous tumor located within a bodily tissue. In other embodiments, the control circuitry 1006 can be configured to direct the electric field generating circuit 1020 to deliver an electric field via the housing 1062 of medical device 402 to the site of a cancerous tumor located within a bodily tissue. In other embodiments, the control circuitry 1006 can be configured to direct the electric field generating circuit 1020 to deliver an electric field between leads 506 and the electrodes thereon and the housing 1062 of medical device 402. In some embodiments, one or more leads 506 and the electrodes thereon can be in electrical communication with the electric field generating circuit 1020.

In some embodiments, medical device 402 can include an electric field sensing circuit 1022 configured to generate a signal corresponding to sensed electric fields. Electric field sensing circuit 1022 can be integrated with control circuitry 1006 or it can be separate from control circuitry 1006.

In some embodiments, sensing electrodes (not shown in this view) can be disposed on or adjacent to the housing of the medical device, on one or more leads connected to the housing, on a separate device implanted near or in the tumor, or any combination of these locations. In some embodiments, the electric field sensing circuit 1022 can measure the electrical potential difference (voltage) between a first electrode and a second electrode, wherein the first and second electrodes are in any of the aforementioned locations. In some embodiments, the electric field sensing circuit can be configured to measure sensed electric fields and to record electric field strength in V/cm.

In some embodiments, the one or more leads 506 can be in electrical communication with the electric field generating circuit 1020. The one or more leads 506 can include one or more electrodes. In some embodiments, various electrical conductors, such as electrical conductors 1026 and 1028, can pass from the header 1044 through a feed-through structure 1030 and into the interior volume 1002 of medical device 402. As such, the electrical conductors 1026 and 1028 can serve to provide electrical communication between the one or more leads 506 and control circuitry 1006 disposed within the interior volume 1002 of the housing 1062.

In some embodiments, recorder circuitry can be configured to record the data produced by the electric field sensing circuit 1022 and record time stamps regarding the same. In some embodiments, the control circuitry 1006 can be hardwired to execute various functions, while in other embodiments the control circuitry 1006 can be directed to implement instructions executing on a microprocessor or other external computation device. A wireless communication interface can also be provided for communicating with external computation devices such as a programmer, a home-based unit, and/or a mobile unit (e.g., a cellular phone, personal computer, smart phone, tablet computer, smartwatch, and the like).

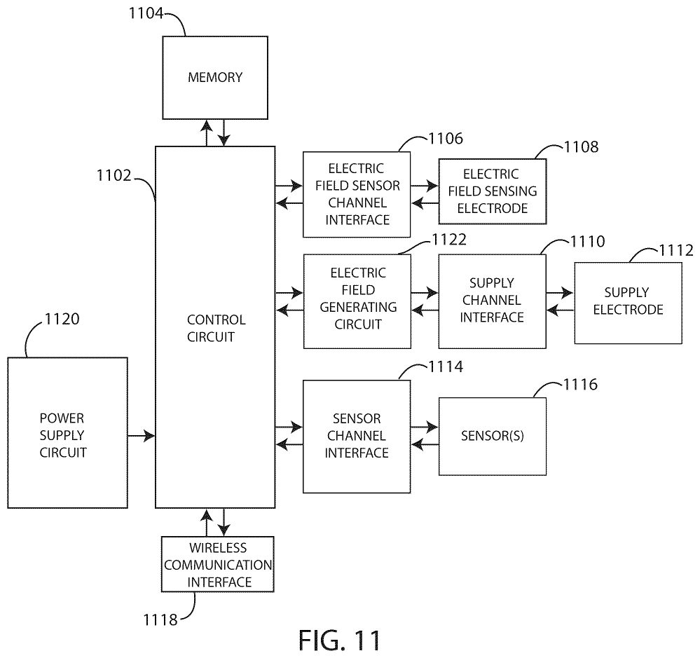

Elements of various embodiments of the medical devices described herein are shown in FIG. 11. However, it will be appreciated that some embodiments can include additional elements beyond those shown in FIG. 11. In addition, some embodiments may lack some elements shown in FIG. 11. The medical devices as embodied herein can gather information through one or more sensing channels and can output information through one or more field generating channels. A microprocessor 1102 can communicate with a memory 1104 via a bidirectional data bus. The memory 1104 can include read only memory (ROM) or random-access memory (RAM) for program storage and RAM for data storage. The microprocessor 1102 can also be connected to a wireless communication interface 1118 for communicating with external devices such as a programmer, a home-based unit and/or a mobile unit (e.g., a cellular phone, personal computer, smart phone, tablet computer, and the like) or directly to the cloud or another communication network as facilitated by a cellular or other data communication network. The medical device can include a power supply circuit 1120. In some embodiments, the medical device can include an inductive energy receiver coil interface (not shown) communicatively coupled or attached thereto to facilitate transcutaneous recharging of the medical device.

The medical device can include one or more electric field sensing electrodes 1108 and one or more electric field sensor channel interfaces 1106 that can communicate with a port of microprocessor 1102. The medical device can also include one or more electric field generating circuits 1122, one or more supply electrodes 1112, and one or more supply channel interfaces 1110 that can communicate with a port of microprocessor 1102. The medical device can also include one or more sensors 1116 (such as temperature sensors) and one or more sensor channel interfaces 1114 that can communicate with a port of microprocessor 1102. The channel interfaces 1106, 1110, and 1114 can include various components such as analog-to-digital converters for digitizing signal inputs, sensing amplifiers, registers which can be written to by the control circuitry in order to adjust the gain and threshold values for the sensing amplifiers, source drivers, modulators, demodulators, multiplexers, and the like.

In some embodiments, one or more physiological sensors can also be included herein. In some embodiments, the physiological sensors can include sensors that monitor temperature, blood flow, blood pressure, and the like. In some embodiments, the respiration sensors can include sensors that monitor respiration rate, respiration peak amplitude, and the like. In some embodiments, the chemical sensors can measure the quantity of an analyte present in a treatment area about the sensor, including but not limited to analytes such as of blood urea nitrogen, creatinine, fibrin, fibrinogen, immunoglobulins, deoxyribonucleic acids, ribonucleic acids, potassium, sodium, chloride, calcium, magnesium, lithium, hydronium, hydrogen phosphate, bicarbonate, and the like. However, many other analytes are also contemplated herein. Exemplary chemical/analyte sensors are disclosed in commonly owned U.S. Pat. No. 7,809,441 to Kane et al., and which is hereby incorporated by reference in its entirety.

Although the temperature sensors 1116 are shown as part of a medical device in FIG. 11, it is realized that in some embodiments one or more of the sensors could be physically separate from the medical device. In various embodiments, one or more of the can be within another implanted medical device communicatively coupled to a medical device via wireless communication interface 1118. In yet other embodiments, one or more of the sensors can be external to the body and coupled to a medical device via wireless communication interface 1118.

### Electric Field Therapy Parameters

In some embodiments, medical devices herein can generate one or more electric fields between selected pairs of electrodes at frequencies selected from a range of between 10 kHz to 1 MHz. In some embodiments, the one or more electric fields can be effective to prevent and/or disrupt cellular mitosis in a cell. In some embodiments, the one or more electric fields can be effective to prevent and/or disrupt cellular mitosis in a cell, but not cause tissue ablation. In some embodiments, the system can be configured to deliver an electric field at one or more frequencies selected from a range of within 300 kHz to 500 kHz. In some embodiments, the system can be configured to deliver an electric field at one or more frequencies selected from a range of within 100 kHz to 300 kHz. In some embodiments, the system can be configured to periodically deliver an electric field using one or more frequencies greater than 10 kHz.

A desired electric field strength can be achieved by delivering an electric current between two electrodes. The specific current and voltage at which the electric field is delivered can vary and can be adjusted to achieve the desired electric field strength at the site of the tissue to be treated. In some embodiments, the system can be configured to deliver an electric field using currents ranging from 1 mAmp to 1000 mAmp to the site of a cancerous tumor. In some embodiments, the system can be configured to deliver an electric field using currents ranging from 20 mAmp to 500 mAmp to the site of a cancerous tumor. In some embodiments, the system can be configured to deliver an electric field using currents ranging from 30 mAmp to 300 mAmp to the site of a cancerous tumor.

In some embodiments, the system can be configured to deliver an electric field using currents including 1 mAmp, 2 mAmp, 3 mAmp, 4 mAmp, 5 mAmp, 6 mAmp, 7 mAmp, 8 mAmp, 9 mAmp, 10 mAmp, 15 mAmp, 20 mAmp, 25 mAmp, 30 mAmp, 35 mAmp, 40 mAmp, 45 mAmp, 50 mAmp, 60 mAmp, 70 mAmp, 80 mAmp, 90 mAmp, 300 mAmp, 125 mAmp, 150 mAmp, 175 mAmp, 400 mAmp, 225 mAmp, 250 mAmp, 275 mAmp, 300 mAmp, 325 mAmp, 350 mAmp, 375 mAmp, 400 mAmp, 425 mAmp, 450 mAmp, 475 mAmp, 500 mAmp, 525 mAmp, 550 mAmp, 575 mAmp, 600 mAmp, 625 mAmp, 650 mAmp, 675 mAmp, 700 mAmp, 725 mAmp, 750 mAmp, 775 mAmp, 800 mAmp, 825 mAmp, 850 mAmp, 875 mAmp, 900 mAmp, 925 mAmp, 950 mAmp, 975 mAmp, or 1000 mAmp. It will be appreciated that the system can be configured to deliver an electric field at a current falling within a range, wherein any of the forgoing currents can serve as the lower or upper bound of the range, provided that the lower bound of the range is a value less than the upper bound of the range.

In some embodiments, the system can be configured to deliver an electric field using voltages ranging from 1 Vrms to 50 Vrms to the site of a cancerous tumor. In some embodiments, the system can be configured to deliver an electric field using voltages ranging from 5 Vrms to 30 Vrms to the site of a cancerous tumor. In some embodiments, the system can be configured to deliver an electric field using voltages ranging from 10 Vrms to 20 Vrms to the site of a cancerous tumor.

In some embodiments, the system can be configured to deliver an electric field using one or more voltages including 1 Vrms, 2 Vrms, 3 Vrms, 4 Vrms, 5 Vrms, 6 Vrms, 7 Vrms, 8 Vrms, 9 Vrms, 10 Vrms, 15 Vrms, 20 Vrms, 25 Vrms, 30 Vrms, 35 Vrms, 40 Vrms, 45 Vrms, or 50 Vrms. It will be appreciated that the system can be configured to deliver an electric field at a voltage falling within a range, wherein any of the forgoing voltages can serve as the lower or upper bound of the range, provided that the lower bound of the range is a value less than the upper bound of the range.

In some embodiments, the system can be configured to deliver an electric field using one or more frequencies including 10 kHz, 20 kHz, 30 kHz, 40 kHz, 50 kHz, 60 kHz, 70 kHz, 80 kHz, 90 kHz, 300 kHz, 125 kHz, 150 kHz, 175 kHz, 400 kHz, 225 kHz, 250 kHz, 275 kHz, 300 kHz, 325 kHz, 350 kHz, 375 kHz, 400 kHz, 425 kHz, 450 kHz, 475 kHz, 500 kHz, 525 kHz, 550 kHz, 575 kHz, 600 kHz, 625 kHz, 650 kHz, 675 kHz, 700 kHz, 725 kHz, 750 kHz, 775 kHz, 800 kHz, 825 kHz, 850 kHz, 875 kHz, 900 kHz, 925 kHz, 950 kHz, 975 kHz, 1 MHz. It will be appreciated that the system can be configured to deliver an electric field using a frequency falling within a range, wherein any of the foregoing frequencies can serve as the upper or lower bound of the range, provided that the upper bound is greater than the lower bound.

In some embodiments, the system can be configured to generate one or more applied electric field strengths selected from a range of within 0.25 V/cm to 1000 V/cm, or 0.25 V/cm to 500 V/cm, or 0.25 V/cm to 100 V/cm, or 0.25 V/cm to 50 V/cm. In some embodiments, the system can be configured to generate one or more applied electric field with strengths of greater than 3 V/cm. In some embodiments, the system can be configured to generate one or more applied electric field strengths selected from a range of within 1 V/cm to 10 V/cm. In some embodiments, the system can be configured to generate one or more applied electric field strengths selected from a range of within 3 V/cm to 5 V/cm.

In other embodiments, the system can be configured to deliver one or more applied electric field strengths including 0.25 V/cm, 0.5 V/cm, 0.75 V/cm, 1.0 V/cm, 2.0 V/cm, 3.0 V/cm, 5.0 V/cm, 6.0 V/cm, 7.0 V/cm, 8.0 V/cm, 9.0 V/cm, 10.0 V/cm, 20.0 V/cm, 30.0 V/cm, 40.0 V/cm, 50.0 V/cm, 60.0 V/cm, 70.0 V/cm, 80.0 V/cm, 90.0 V/cm, 300.0 V/cm, 125.0 V/cm, 150.0 V/cm, 175.0 V/cm, 400.0 V/cm, 225.0 V/cm, 250.0 V/cm, 275.0 V/cm, 300.0 V/cm, 325.0 V/cm, 350.0 V/cm, 375.0 V/cm, 400.0 V/cm, 425.0 V/cm, 450.0 V/cm, 475.0 V/cm, 500.0 V/cm, 600.0 V/cm, 700.0 V/cm, 800.0 V/cm, 900.0 V/cm, 1000.0 V/cm. It will be appreciated that the system can generate an electric field having a field strength at a treatment site falling within a range, wherein any of the foregoing field strengths can serve as the upper or lower bound of the range, provided that the upper bound is greater than the lower bound.

In some embodiments, an electric field can be applied to the site of a cancerous tumor or tumor resection at a specific frequency or constant frequency range.

### Methods

Many different methods are contemplated herein, including, but not limited to, methods of treating pancreatic cancer, methods of treating unresectable tumors, methods of delivery cancer therapy, methods of placing electrical stimulation leads, and the like. Aspects of system/device operation described elsewhere herein can be performed as operations of one or more methods in accordance with various embodiments herein.

In some embodiments, operations described herein and method steps can be performed as part of a computer-implemented method executed by one or more processors of one or more computing devices. In various embodiments, operations described herein and method steps can be implemented instructions stored on a non-transitory, computer-readable medium that, when executed by one or more processors, cause a system to execute the operations and/or steps.

In an embodiment, a method of treating pancreatic cancer is included. The method can include inserting an electrical stimulation lead through the inferior vena cava, a hepatic vein, and into the portal vein via a transjugular intrahepatic portosystem shunt (TIPS). Then the electrical stimulation lead can be inserted into at least one of the superior mesenteric vein and the splenic vein. The method can also include positioning electrodes on the lead into at least one of the superior mesenteric vein and the splenic vein. The method can also include delivering one or more electric fields through the electrodes to a treatment zone including at least a portion of the pancreas. The electric fields can be effective to prevent and/or disrupt cellular mitosis in a cell.

In an embodiment, the method can further include implanting a medical device configured to generate the one or more electrical fields, the medical device including control circuitry and a therapy output circuit. The method can further include attaching the electrical stimulation lead to the medical device.

In an embodiment, the medical device further can include a conductive housing. The conductive housing can be configured to serve as an electrode for use with delivering the one or more electrical fields.

In an embodiment of the method, inserting the electrical stimulation lead can include inserting a first electrical stimulation lead into the superior mesenteric vein and inserting a second electrical stimulation lead into the splenic vein.

While cancers of various types can be treated, embodiments herein can be particularly useful to treat unresectable pancreatic cancer tumors. In an embodiment, the method can further include selecting a patient with an unresectable pancreatic tumor. In an embodiment, the method can further include selecting a patient with tumor ingrowth into the superior mesenteric artery or celiac axis.

In an embodiment, the method can further include selecting one or more pairs of electrodes forming a vector across which to deliver electrical fields so as to include a target treatment zone, such as including a pancreatic tumor.

In an embodiment, a method of treating pancreatic cancer is included herein that includes inserting an electrical stimulation lead into the inferior vena cava and positioning electrodes on the lead within the inferior vena cava and adjacent to the pancreas. The method can further include delivering one or more electric fields through the electrodes to a treatment zone including at least a portion of the pancreas, wherein the electric fields are effective to prevent and/or disrupt cellular mitosis in a cell.

In an embodiment, the method can further include implanting a medical device configured to generate the one or more electrical fields, the medical device can include control circuitry and a therapy output circuit. The method can further include attaching the electrical stimulation lead to the medical device.

In an embodiment, the method can further include inserting a second electrical stimulation lead transversely across the abdomen. The second electrical stimulation lead can include a second set of electrodes disposed thereon.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A method of treating pancreatic cancer comprising:
inserting an electrical stimulation lead through the inferior vena cava, a hepatic vein, and into the portal vein via a transjugular intrahepatic portosystem shunt (TIPS), then into at least one of the superior mesenteric vein and the splenic vein;
positioning electrodes connected to the lead into at least one of the superior mesenteric vein and the splenic vein; and
delivering one or more electric fields through the electrodes to a treatment zone including at least a portion of the pancreas, wherein the one or more electric fields are at frequencies selected from a range of between 10 kHz to 1 MHz, wherein the electric fields are effective to prevent and/or disrupt cellular mitosis in a cell.

2. The method of treating pancreatic cancer of claim 1, further comprising:
implanting a medical device configured to generate the one or more electrical fields, the medical device comprising control circuitry and a therapy output circuit; and
attaching the electrical stimulation lead to the medical device.

3. The method of treating pancreatic cancer of claim 2, the medical device further comprising a conductive housing, wherein the conductive housing is configured to serve as an electrode for use with delivering the one or more electrical fields.

4. The method of treating pancreatic cancer of claim 1, wherein inserting the electrical stimulation lead comprises:
inserting a first electrical stimulation lead into the superior mesenteric vein; and
inserting a second electrical stimulation lead into the splenic vein.

5. The method of treating pancreatic cancer of claim 1, wherein the one or more electric fields are at a field strength of 1 V/cm to 10 V/cm within the treatment zone.

6. The method of treating pancreatic cancer of claim 1, further comprising selecting a patient with an unresectable pancreatic tumor.

7. The method of treating pancreatic cancer of claim 1, further comprising selecting a patient with tumor ingrowth into the superior mesenteric artery or celiac axis.

8. The method of treating pancreatic cancer of claim 1, wherein at least one of the electrodes has an axial length from 1.5 to 2.5 centimeters.

9. The method of treating pancreatic cancer of claim 1, wherein at least one of the electrodes has a diameter of 0.8 to 1.2 millimeters.

10. The method of treating pancreatic cancer of claim 1, further comprising selecting one or more pairs of electrodes forming a vector across which to deliver electrical fields.

11. A method of treating pancreatic cancer comprising:
inserting an electrical stimulation lead into the inferior vena cava;
positioning electrodes connected to the lead within the inferior vena cava and adjacent to the pancreas; and
delivering one or more electric fields through the electrodes to a treatment zone including at least a portion of the pancreas, wherein the one or more electric fields are at frequencies selected from a range of between 10 kHz to 1 MHz, wherein the electric fields are effective to prevent and/or disrupt cellular mitosis in a cell.

12. The method of treating pancreatic cancer of claim 11, further comprising:
implanting a medical device configured to generate the one or more electrical fields, the medical device comprising control circuitry and a therapy output circuit; and
attaching the electrical stimulation lead to the medical device.

13. The method of treating pancreatic cancer of claim 12, the medical device further comprising a conductive housing, wherein the conductive housing is configured to serve as an electrode for use with delivering the one or more electrical fields.

14. The method of treating pancreatic cancer of claim 11, wherein the one or more electric fields are at a field strength of 1 V/cm to 10 V/cm within the treatment zone.

15. The method of treating pancreatic cancer of claim 11, further comprising:
inserting a second electrical stimulation lead transversely across the abdomen, the second electrical stimulation lead comprising a second set of electrodes disposed thereon.

16. The method of treating pancreatic cancer of claim 11, further comprising selecting a patient with an unresectable pancreatic tumor.

17. The method of treating pancreatic cancer of claim 11, further comprising selecting a patient with tumor ingrowth into the superior mesenteric artery or celiac axis.

18. The method of treating pancreatic cancer of claim 11, wherein at least one of the electrodes has an axial length from 1.5 to 2.5 centimeters.

19. The method of treating pancreatic cancer of claim 11, wherein at least one of the electrodes has a diameter of 0.8 to 1.2 millimeters.

20. A method of treating pancreatic cancer comprising:
inserting electrodes into a patient;
positioning the electrodes into a ductal system of a pancreas of the patient; and
delivering one or more electric fields through the electrodes to a treatment zone including at least a portion of the pancreas, wherein the one or more electric fields are at frequencies selected from a range of between 10 kHz to 1 MHz, wherein the electric fields are effective to prevent and/or disrupt cellular mitosis in a cell.
